(19) United States
(12) Patent Application Publication
(10) Pub. No.: US 2022/0273515 A1
(43) Pub. Date: Sep. 1, 2022

(54) MEDICAL AMBULATORY AID

(71) Applicant: CURT TUCKER, SAGINAW, MI (US)

(72) Inventor: CURT TUCKER, SAGINAW, MI (US)

(21) Appl. No.: 17/663,510

(22) Filed: May 16, 2022

Related U.S. Application Data
(63) Continuation-in-part of application No. 16/804,186, filed on Feb. 28, 2020.

(51) Int. Cl. A61H 3/00

(52) U.S. Cl. CPC A61H 3/008; A61H 2003/007; A61H 2201/1642

(57) ABSTRACT

A medical ambulatory aid comprising, in combination, a waist belt harness, a thigh brace, a calf brace and a walking boot.

### CROSS-REFERENCE TO RELATED APPLICATIONS

This application is a continuation in part of U.S. application Ser. No. 16/804,186 filed on Feb. 20, 2020.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

The present invention is a medical ambulatory aid. The applicant is aware of the following prior art.

United States patent publication 2015/0335515 to lee is directed to an electrical walking aid that uses a waist belt. However, the ischial straps of the Lee device are for stability of the waist portion of the device so that the waist portion stays around the patient's waist, and so that the patient does not slide down through the waist belt to the floor. The ischial strap labeled 12R, 12L, 16R and 16L in FIG. 5A in the drawings do not provide any weight bearing or relief for the patient. The Lee device does not have a one button, quick release mechanism either.

U.S. patent Publication 2016/0128890 to LaChappelle is a bilateral device unlike the device of the instant invention which is unilateral. The upright portion of the brace would be unable to support the impaired limb and no support system of straps is shown in LaChappelle.

Bledsoe, U.S. Patent publication 2005/0131324 shows only a foot/ankle portion of a brace.

Chan, U.S. Patent publication 2009/0306565 also shows only a foot/ankle portion of a brace. That is, the Chan device is only an external portion of a walking boot which is extremely similar to the external portion of the Bledsoe walking boot device.

U.S. Pat. No. 6,010,474 entitled, Orthopedic brace for legs, issued to Wycoki on Jan. 4, 2000, teaches a medical brace that is arranged to transfer weight bearing, during walking, above the patient's knee to the patient's thigh and pelvic infrastructure. This patent does not teach a device that uses a waist belt and under-leg strap. Therefore, Wycoki does not have the utility that the present invention has. The present invention transfers all the weight to the thigh and pelvic infrastructure teaching a more complete and comprehensive transfer of weight from the knee, lower leg and foot areas than Wycoki does.

U.S. Pat. No. 10,434,003 entitled, lower extremity isolating leg brace, issued to Robinson on Oct. 8, 2019, does incorporate a waist band. However, its buckle is not quick release which helps the patient with easy removal and application. Also, Robinson does not teach the under-leg strap. The present invention teaches the under-leg strap which holds the weight that is transferred to the pelvic region.

### BRIEF SUMMARY OF THE INVENTION

The present invention is a medical ambulatory aid. The medical ambulatory aid comprises in combination a waist belt harness, a thigh brace, a calf brace and a walking boot. The word “near” as used herein is equivalent to “proximal”.

There is also a heel strap detachedly attached to the walking boot. There is a metal assembly supporting the waist belt harness, a thigh brace, a calf brace, and a walking boot. This assembly comprises a first metal bar detachedly attached to a side wall of the walking boot at a distal end of the first metal bar. There is surmounted on a near end of the first metal bar, a second metal bar that has a distal end. There is also surmounted on the distal end of the second metal bar, a calf U-channel that has a distal end. The calf brace is attached to the near end of the calf U-channel.

There is a third metal bar that is attached to the near end of the second metal bar.

There is a knee hinge surmounted on the near end of the third bar. There is a fourth metal bar surmounted on a near end of the knee hinge. There is a thigh U-channel surmounted on a near end of the fourth metal bar.

There is a thigh brace mounted on the thigh U-channel.

There is a fifth metal bar surmounted on a near end of the thigh U-channel. There is also a hip hinge surmounted on a near end of the fifth metal bar.

There is a waist belt harness attached to a near end of the hip hinge. The waist belt harness is comprised of a waist belt that is configured to rest on a wearer's ilium bone. The waist belt harness has a one button belt release buckle attached thereto and an under-leg body weight bearing strap that is configured to wrap around a wearer's gluteus maximus muscles and ischium bone.

### BRIEF DESCRIPTION OF THE SEVERAL VIEWS OF THE DRAWING(S)

FIG. 1 shows the medical ambulatory aid of this invention.

FIG. 2 shows the medical ambulatory aid waist belt harness and thigh brace of FIG. 1.

FIG. 3 shows the medical ambulatory aid calf brace of FIG. 1.

FIG. 4 shows the medical ambulatory aid calf brace and walking boot of FIG. 1.

### DETAILED DESCRIPTION OF THE INVENTION AND DRAWINGS

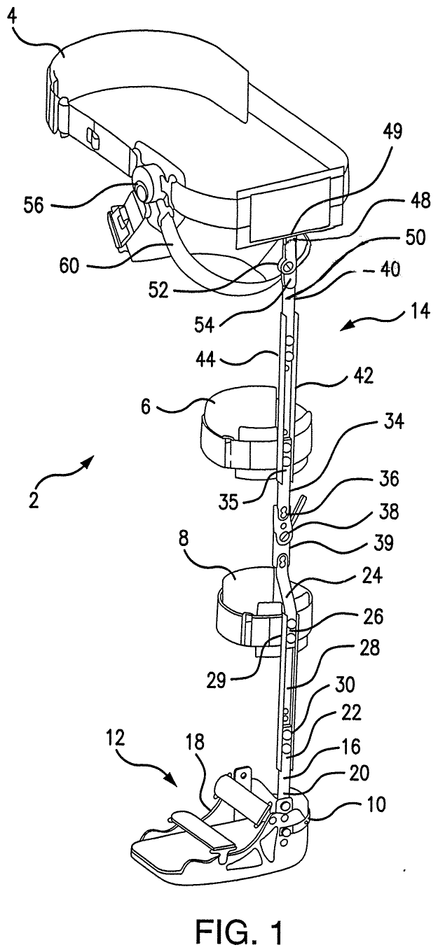

FIG. 1 shows the medical ambulatory aid. The present invention is a medical ambulatory aid 2. The medical ambulatory aid 2 comprises in combination a waist belt harness 4, a thigh brace 6, a calf brace 8 and a walking boot 12.

There is a heel strap 10 detachedly attached to the walking boot 12. There is a metal assembly 14 supporting the waist belt harness 4, a thigh brace 6, a calf brace 8 and the walking boot 12. This assembly 14 comprises a first metal bar 16 detachedly attached to a side wall 18 of the walking boot 12 at a distal end 20 of the first metal bar 16. There is surmounted on a near end 22 of the first metal bar 16, a calf U-channel 28. There is a second metal bar 24 that has a distal end 26. There is also surmounted on the distal end 26 of the second metal bar 24, a calf U-channel 28 that has a distal end 30. The calf brace 32 is attached to the near end 29 of the calf U-channel 28.

There is a third metal bar 34 that is attached to the near end 36 of the knee hinge 38.

There is a knee hinge 38 surmounted on the near end 36 of the third bar 34. There is a thigh U-channel 42 surmounted on a near end 39 of the knee hinge 38 and near end 35 of the U-channel 42. There is a thigh U-channel 42 surmounted on a near end 44 of the fourth metal bar 40.

There is a thigh brace 6 mounted on the thigh U-channel 42.

There is a fifth metal bar 48 surmounted on a distal end 50 of the thigh U-channel 42. The fifth metal bar 48 has a near end 49. There is also a hip hinge 52 surmounted on a near end 50 of the fifth metal bar 48.

There is a waist belt harness 4 attached to a near end 49 of the fifth metal bar 48, this waist belt harness 4 comprised of a waist belt 4 that is configured to rest on a wearer's ilium bone. The waist belt harness 4 has a one-button quick release belt and buckle mechanism 56 attached thereto and a body weight bearing under-leg strap 60 that is configured to wrap around a wearer's gluteus maximus muscles and ischium bone.

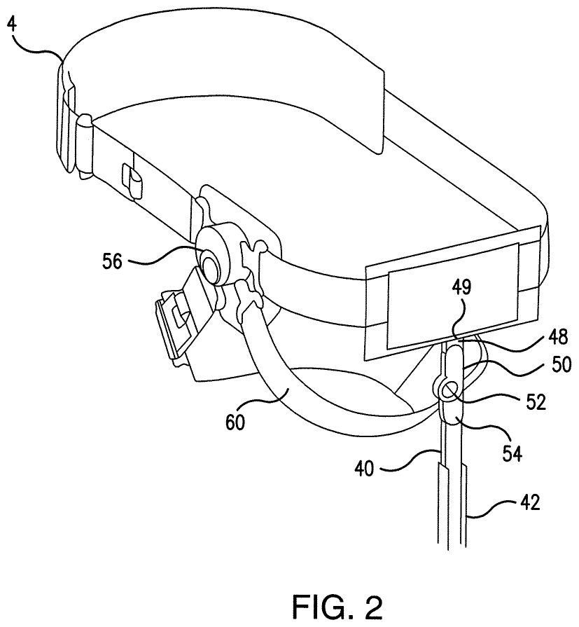

FIG. 2 shows the medical ambulatory aid waist belt harness 4. The is a waist belt harness 4 attached to a distal end 53 of the hip hinge 52, this waist belt harness 4 comprised of a waist belt 4 that is configured to rest on a wearer's ilium bone. The waist belt harness 4 has a one-button quick belt and buckle release mechanism 56 attached thereto and a body weight bearing under-leg strap 60 that is configured to wrap around a wearer's gluteus maximus muscles and ischium bone. It has been discovered that the waist belt harness 4, release mechanism 56 and the under-leg strap provides a hands-free ambulatory aid that reduces the compressive load on the knee and acetabulofemoral joints, to quicken the recovery process and mitigate the transition from surgery to full recovery after leg, hip, knee and bone impairment.

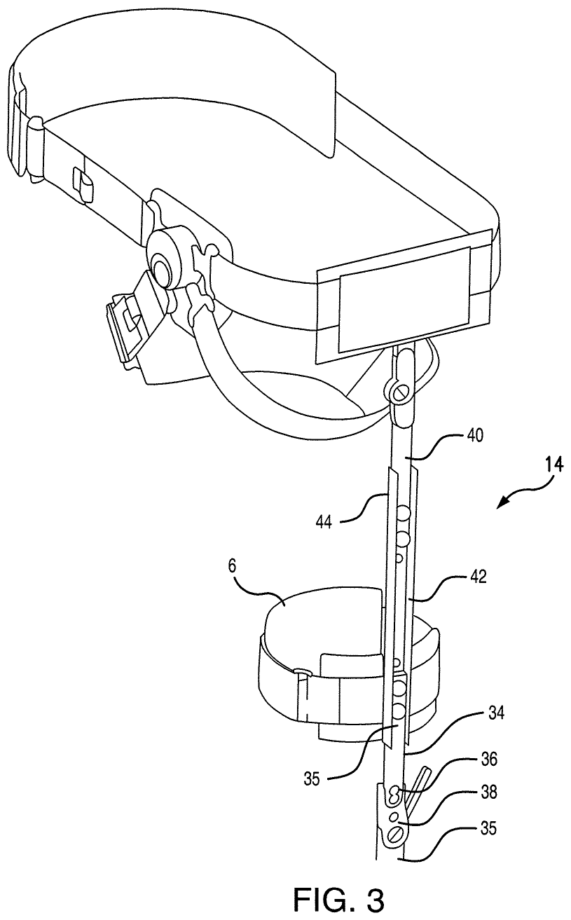

FIG. 3 shows the medical ambulatory aid thigh brace 8. This thigh brace 8 encircles the thigh and is tightened to secure the ambulatory aid to it. The fourth metal bar 40 is attached to the thigh U-channel 42 at the metal bar 40 near end 44. The thigh brace 6 is attached to the thigh U-channel 42. The thigh brace 6 provides thigh support for the walking ambulatory aid 2.

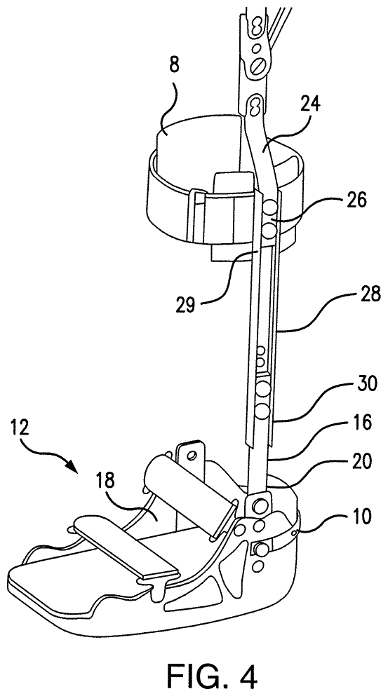

FIG. 4 shows the medical ambulatory aid 2, calf brace 8 and walking boot 12. There is surmounted on the distal end 26 of the second metal bar 24, a calf U-channel 28 that has a distal end 30. The calf brace 32 surrounds and supports the calf area. The calf brace 32 is attached to the near end 30 of the calf U-channel 28. The first metal bar 16 detachedly attached to a side wall 18 of the walking boot 12 at a distal end 20 of the first metal bar 16. The walking boot 12 also has a heel strap 10. The heel strap 10 keeps the user's foot in place in the walking boot during movement. The device is configured so that all of the body weight of the wearer can be supported on the under-leg strap 60. There is surmounted on a near end 22 of the first metal bar 16, a second metal bar 24 that has a distal end 26. There is also surmounted on the distal end 26 of the second metal bar 24, a calf U-channel 28 that has a near end 30. The calf brace 32 is attached to the near end 30 of the calf U-channel 28.

The present invention incorporates a one-button quick release buckle for ease of release for the waist strap and under-leg strap. It has been discovered that the waist belt and under-leg strap give a higher level of weight transfer from the knee, lower leg or calf and foot areas than other devices that either have neither or just one component. The present invention teaches the under-leg strap that holds the weight that is transferred to the pelvic region. This is the essence of the present invention. The weight transfer provided by the under-leg strap that is configured to wrap around a wearer's gluteus maximus muscles and ischium bone, transfers the weight.

The ability of the present device to place the weight in the pelvic region provides a greater utility over all prior art inventions.

What is claimed is:

1. A medical ambulatory aid, said medical ambulatory aid consisting of in combination:
(I) a waist belt harness having a one-button quick release buckle;
(II) a thigh brace;
(III) a calf brace;
(IV) a walking boot
(V) a heel strap detachedly attached to said walking boot;
(VI) a metal assembly supporting (I)-(V), said metal assembly comprising:
a. a first metal bar detachedly attached to a side wall of said walking boot at a distal end of said first metal bar;
b. surmounted on a proximal end of said first metal bar, a second metal bar having a distal end;
c. surmounted on said distal end of said second metal bar, a calf U-channel having a distal end;
d. said calf brace being attached to a proximal end of said calf U-channel;
e. a third metal bar being attached to a proximal end of said second metal bar;
f. a knee hinge surmounted on a distal end of said third bar;
g. a fourth metal bar surmounted on a proximal end of said knee hinge;
h. a thigh U-channel mounted on a proximal end of said fourth metal bar;
i. said thigh brace mounted on said thigh U-channel;
j. a fifth metal bar surmounted on a proximal end of said thigh U-channel;
k. a hip hinge surmounted on a distal end of said fifth metal bar;
l. said waist belt harness attached to a proximal end of said hip hinge, said waist belt harness comprised of:
(i) a waist belt that is configured to rest on a wearer's ilium bone, said waist belt harness having said belt with a one-button quick release buckle attached thereto, and,
(ii) an under-leg body weight bearing strap that is configured to wrap around a wearer's gluteus maximus muscles and ischium bone and support the body weight of a wearer such that an enclosed foot of a wearer does not touch a supporting surface.
